(19) United States
(12) Patent Application Publication
(10) Pub. No.: US 2004/0038258 A1
(43) Pub. Date: Feb. 26, 2004

(54) Methods for detecting DNA polymorphisms

(76) Inventors: John B. Harley, Oklahoma City, OK (US); Kenneth M. Kaufman, Oklahoma City, OK (US)

Correspondence Address: Steven L. Highlander, FULBRIGHT & JAWORSKI L.L.P., Suite 2400, 600 Congress Avenue, Austin, TX, 78701-3271, US

(21) Appl. No.: 10/407,846

(22) Filed: Apr. 4, 2003

Related U.S. Application Data
(60) Provisional application No. 60/376,360, filed on Apr. 29, 2002.

(51) Int. Cl. C12Q 1/68; C12P 19/34

(52) U.S. Cl. 435/6; 435/91.2

(57) ABSTRACT

The present invention provides methods for the detection of polymorphisms. The methods rely on the omission of a single base from a polymerization cocktail such that, during nucleic acid synthesis, the absence of a particular base will discriminate between different forms of a polymorphism, the identity of which is determined by the length of the synthesized product. Kits for carrying out these methods also are provided.

The present invention claims benefit of priority to U.S. Provisional Serial No. 60/376,360, filed Apr. 23, 2002, the entire contents of which are hereby incorporated by reference.

### BACKGROUND OF THE INVENTION

1. Field of the Invention

The present invention relates to the fields of molecular biology and genetics. More specifically, the invention relates to methods of detecting DNA polymorphisms using primers selected for their proximity to the polymorphisms, and cocktails of no more than three nucleotide triphosphates (NTPs) which include only one of the possible bases located at the polymorphism site.

2. Related Art

There are numerous sequence variations in the deoxyribonucleic acid (DNA) among individual members of a species. These variations, also termed polymorphisms, may be insertions, deletions, inversions, variable repeats, and substitutions of DNA nucleotides. Single nucleotide polymorphisms (SNPs) are the most common chromosomal differences between individual human beings. According to the NCBI's SNP database, SNPs occur every 100 to 300 nucleotide base pairs of human DNA.

Methods to reliably detect genetic variations have a myriad of applications. For example, it is increasingly apparent that polymorphisms are valuable genetic markers for determining identity, establishing genetic linkages, and for predicting, diagnosing and treating disease. In the field of pharmacogenomics, for instance, an individual's genetic profile may be used to customize medical treatment based on the individual's ability to metabolize and tolerate specific drugs. These methods have application in any field related to biology with application to life in any form including agriculture, environmental sciences, marine sciences, medicine, paleontology, and others.

Even though knowledge of genetic polymorphisms can reveal important differences between individuals, methods of detecting polymorphisms must be reliable, rapid, and inexpensive in order to have practical utility. Currently, there is an acute need for simple procedures that can rapidly and inexpensively provide a reliable method to detect genetic differences between individuals.

### SUMMARY OF THE INVENTION

Thus, in accordance with the present invention, there is provided a method for identifying a nucleic acid polymorphism comprising (a) providing a target polynucleotide comprising a first polymorphic site; (b) providing a first oligonucleotide that hybridizes upstream of the first polymorphic site, the space between the ‘3 end of the first oligonucleotide and the first polymorphic site defined as a first intervening region, wherein the first intervening region lacks one of the bases that occurs at the first polymorphic site; (c) mixing the first oligonucleotide and the polynucleotide with a polymerase and a polymerization cocktail of no more than three different NTPs (rNTPs or dNTPs), wherein the cocktail lacks an NTP for the same base that is missing from the intervening region; (d) subjecting the mixture to conditions that support polymerization; and (e) assessing the length of polymerization products from step (d), wherein a polymerization product equal in size to the first oligonucleotide plus the first intervening region indicates that polymerization terminated at the polymorphic site, and that the first polymorphic site contains the base from the NTP lacking from the polymerization cocktail, and wherein a polymerization product greater in size than the first oligonucleotide plus the first intervening region indicates that polymerization continued past the first polymorphic site, and that the first polymorphic site contains a base from an NTP present in the polymerization cocktail. If the target polynucleotide is haploid or homozygous, then a single product will be generated, the length and sequence of which is determined by the polymorphism present. If the target polynucleotide is diploid in the sample being evaluated, then more than one product may be generated, depending on whether the target polynucleotide contains only one of the polymorphisms (a single product), or contains a second form of the polymorphism (two products). The first oligonucleotide may be labeled with a radioisotope, a fluorophore, a chromophore, a dye, or an enzyme.

The method may further comprise providing a second oligonucleotide that hybridizes upstream of a second polymorphic site, the space between the ‘3 end of the second oligonucleotide and the second polymorphic site defined as the second intervening region; wherein the second intervening region lacks one of the bases that occurs at the first and second polymorphic sites, wherein a polymerization product equal in size to the second oligonucleotide plus the second intervening region indicates that polymerization terminated at the second polymorphic site, and that the second polymorphic site contains the base from the NTP lacking from the polymerization cocktail, and wherein a second polymerization product greater in size than the second oligonucleotide plus the second intervening region indicates that polymerization continued past the second polymorphic site, and that the second polymorphic site contains a base from an NTP present in the polymerization cocktail. The second oligonucleotide plus second intervening region may vary in size from the first oliognonucleotide plus first intervening region. The second oligonucleotide may be labeled such that the second polymerization product can be distinguished from the first oliognonucleotide plus first intervening region.

The first oligonucleotide is about 10 to about 10,000 bases in length, about 10 to about 100 bases in length, or 10 to 25 bases in length. That portion of the first oliogonucleotide that hybridizes to the target polynucleotide may be 6 to about 80 bases, 10 to about 80 bases, 10 to about 40 bases, 6 to about 25 bases, or 10 to 25 bases. Assessing the length of the polymerization product may comprise electrophoretic separation, chromatography, and may further comprise staining with silver stain or an intercalating dye. Assessing the length of the polymerization product also may be determined by mass spectroscopy.

The method may also further comprise, prior to step (e) but following step (d), the additional steps of (i) dissociating polymerization products from the target polynucleotide sequence; (ii) annealing second copy of the first oligonucleotide to the target polynucleotide; (iii) mixing the second copy of the first oligonucleotide and the target polynucleotide with a polymerase and a cocktail of no more than three different NTPs, wherein the cocktail lacks a NTP for the same base that is missing from the intervening region; and iv) subjecting the mixture to conditions that support polymerization. Steps (i)-(iv) may be repeated. The polymerase may be a thermostable polymerase. The polymorphic site may comprise a deletion, a single nucleotide polymorphism or an insertion, such as a duplication, inversion or a translocation. The intervening sequence may be 1-50 bases, 2-25 bases, 3-10 bases, or particularly 2, 3, 4, 5, 6, 7, 8, 9 or 10 bases. The cocktail may comprise only two NTPs, and the intervening region contains only the bases corresponding to the two NTP's represented in the cocktail. The cocktail may comprise only one NTP, and the intervening region contains only the base corresponding to the NTP represented in the cocktail.

In another embodiment, there is provided a method for identifying a nucleic acid polymorphism comprising (a) providing a target polynucleotide sequence comprising a first polymorphic site; (b) providing a first oligonucleotide that hybridizes upstream of the polymorphic site, the space between the 3′ end of the first oligonucleotide and the first polymorphic site defined as a first intervening region, wherein the first intervening region lacks one of the bases that occurs at the first polymorphic site; (c) mixing the first oligonucleotide and the polynucleotide with a polymerase and a cocktail of no more than three different NTPs, at least one of which is labeled, wherein the cocktail lacks a NTP for the same base that is missing from the intervening region; (d) subjecting the mixture to conditions that support polymerization; and (e) assessing the length of polymerization products, wherein a polymerization product equal in size to the first oligonucleotide plus the first intervening region indicates that polymerization terminated at the polymorphic site, and that the first polymorphic site contains the base from the NTP lacking from the polymerization cocktail, and wherein a first polymerization product greater in size than the first oligonucleotide plus the first intervening region indicates that polymerization continued past the first polymorphic site, and that the first polymorphic site contains a base from an NTP present in the polymerization cocktail. If the target polynucleotide is haploid, then a single product will be generated, the size of which will be determined by the polymorphism present. If the target polynucleotide is diploid, more than one product may be generated, depending on whether the target polynucleotide contains only one of the polymorphisms (a single product) or contains two forms of the polymorphism (two products). The labeled NTP may be labeled with a radioisotope, a fluorophore, a chromophore, a dye or an enzyme.

The method may further comprise providing a second oligonucleotide that hybridizes upstream of a second polymorphic site, the space between the ‘3 end of the second oligonucleotide and the second polymorphic site defined as the second intervening region; wherein the second intervening region lacks one of the bases that occurs at the first and second polymorphic sites; wherein the second oligonucleotide plus second intervening region varies in size from the first oliognonucleotide plus first intervening region, wherein a polymerization product equal in size to the oligonucleotide plus the second intervening region indicates that polymerization terminated at the second polymorphic site, and that the second polymorphic site contains the base from the NTP lacking from the polymerization cocktail, and wherein a polymerization product greater in size than the oligonucleotide plus the second intervening region indicates that polymerization continued past the second polymorphic site, and that the second polymorphic site contains a base from an NTP present in the polymerization cocktail. Alternatively, a second label, distinguishable from the one used with the first oligonucleotide, may be used to identify the polymorphisms at the second polymorphic site.

The method also may further comprise, prior to step (e) but following step (d), the additional steps of (i) dissociating the polymerization products from the target polynucleotide sequence; (ii) annealing second copy of the first oligonucleotide to the target polynucleotide; (iii) mixing the second copy of the first oligonucleotide and the target polynucleotide with a polymerase and a cocktail of no more than three different NTPs, wherein the cocktail lacks a NTP for the same base that is missing from the intervening region; and (iv) subjecting the mixture to conditions that support polymerization. Steps (i)-(iv) are repeated.

The first oligonucleotide is about 10 to about 10,000 bases in length, 10 to about 100 bases in length or 10 to about 25 bases in length. That portion of the first oliogonucleotide that hybridizes to the target polynucleotide is about 10 to about 80 bases, 10 to about 40 bases, or 10 to 25 bases. Assessing the length of polymerization products may comprise electrophoretic separation, chromatography or mass spectroscopy. The polymerase may be a thermostable polymerase. The polymorphic site may comprise a deletion, an insertion, or a single nucleotide polymorphism. The intervening sequence may be 1-50 bases, 2-25 bases, 3-10 bases or 2, 3, 4, 5, 6, 7, 8, or 10 bases.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein:

FIG. 1—Illustration of the DNA sequence of FcγRIIA in the region of H131R. The A (adenine) to G (guanine) change shown in the DNA sequence causes H131R (histidine to arginine at amino acid position 131). Examples are presented of two forward and two reverse primers that could be used in an embodiment of the invention applied to this single nucleotide polymorphism (SNP). Only one of the four primers (two forward and two reverse are presented) is needed for an embodiment of the invention. Each of the four possibilities is illustrated for this SNP. The predicted products from the reactions that follow are given. The FcγRIIA forward 1 primer reaction uses the 700 nm dye-labeled primer, TCC AGA ATG GAA AAT CCC AG (SEQ ID:NO. 1), and three dNTPs without dGTP. This is predicted to produce three detectable products: the labeled forward primer (Product 1), the primer extended by AAATTCTCCC (SEQ ID:NO. 2)in the “G” allele (this is predicted from the genetic code to make the arginine allele) (Product 2), and the primer extended by AAATTCTCCCATTT (SEQ ID:NO. 3)in the “A” allele predicted to code for the histidine allele) (Product 3). Alternatively, application of the 800 nm dye-labeled reverse primer 1, GTG GGA TGG AGA AGG TGG GAT (SEQ ID:NO. 4), could be extended using three dNTPs without dTTP. Three different species are again detected: the labeled reverse primer (Product 4), the primer extended by CCAAA (SEQ ID:NO. 5) in the “C” allele (this is predicted from the genetic code to make the arginine allele) (Product 5), and the primer extended by CCAAATGGGAGAA (SEQ ID:NO. 6) in the “T” allele (predicted to code for the histidine allele) (Product 6). Alternatively, application of the 700 nm dye-labeled forward primer 2, AGA ATG GAA AAT CCC AGA AA (SEQ ID:NO. 13), could be extended using three dNTPs without dATP. Three different species are again detected: the labeled forward primer 2 (Product 7), the primer extended by TTCTCCCGTTTGG (SEQ ID:NO. 14) in the “G” allele (this is predicted from the genetic code to make the arginine allele) (Product 8), and the primer extended by TTCTCCC (SEQ ID:NO. 15) in the “T” allele (predicted to code for the histidine allele) (Product 9). Alternatively, application of the 800 nm dye-labeled reverse primer 2, GGA TGG AGA AGG TGG GAT CC (SEQ ID:NO. 16), could be extended using three dNTPs without dCTP. Three different species are again detected: the labeled reverse primer 2 (Product 10), the primer extended by AAA in the “C” allele (this is predicted from the genetic code to make the arginine allele) (Product 11), and the primer extended by CCAAATGGGAGAATTT (SEQ ID:NO. 17) in the “T” allele (predicted to code for the histidine allele) (Product 12). Note: still other primers may be chosen that end closer to the polymorphic site, but would also be examples of the invention. All that is required, from a practical standpoint, is that the extension products produced can be distinguished from one another and from the unextended primer, and that the omitted dNTP is not in the sequence between the end of the primer and the polymorphic site.

FIG. 2.—An embodiment wherein the FcγRIIA 700 nm dye-labeled forward primer (FIG. 1) and three dNTPs without dGTP (i.e., dATP, dCPT and dGTP) were used to detect the polymorphisms encoding H131R. Data from 21 subjects were used whose genotype was previously determined by sequencing their DNA were used. The products produced have mobility in polyacrylamide gel electrophoresis (using Li-Cor semi-automatic sequencers in this instance) and have the expected number of base pairs for the two expected alleles, based upon the known sequence (FIG. 1). The primer and both products are labeled. Band 1 corresponds to the “A” allele that encodes histidine at amino acid position 131 and Band 2 corresponds to “G” allele that encodes arginine at amino acid position 131. Data were obtained from all 21 samples, which produced genotypes that agreed exactly with the data obtained when the H131R SNP was sequenced in these same individuals (Fisher exact test p<0.000001). In this preliminary evaluation the sensitivity of the method is 100%, the specificity is 100% and the predictive value is 100% in these samples.

### DETAILED DESCRIPTION OF THE INVENTION

As mentioned above, knowledge of DNA polymorphisms can prove very useful in a variety of applications, including diagnosis and treatment of disease, and may be applied to any virus, bacteria, Archaea, animal or plant. The present invention provides reliable methods for rapidly and inexpensively detecting polymorphisms in DNA sequences. The invention is based on the selection of an oligonucleotide primer that is selected to bind a target DNA sequence upstream of the polymorphism being evaluated, which is termed the target polymorphism. The primer and no more than three NTPs (nucleotide tri-phosphates, either deoxyribose-(d)NTPs or ribose-(r)NTPs) are combined with a polymerase and the target DNA sequence, which serves as a template for amplification. By using less than all four NTPs, it is possible to omit one of the two polymorphic nucleotides needed for incorporation at the polymorphic site. It is important for the practice of the present invention that the amplification be designed such that the omitted nucleotide is not required between the 3′ end of the primer and the target polymorphism. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products based on separation by size/length will reveal which polymorphism is present. The details of these embodiments, as well as others, are described in more detail in the following sections.

I. The Target Polymorphism

The present invention is a method designed to identify differences in DNA sequences, which are also termed polymorphisms. These methods may be adapted to detect the vast majority of DNA substitutions, deletions, insertions, inversions, and variable repeats. If the polymorphism involves multiple differences in the nucleotide sequence, then the choice of the target polymorphism is determined by the ability to distinguish the alleles by, for example, their size. The target nucleic acid may be from a variety of different sources, including genomic DNA, fractionated RNA, whole cell RNA and or nucleic acids expanded by an amplification procedure. The nucleic acid may be from any form of life, but in particular is drawn to animals, such as humans. The source nucleic acid being evaluated may also be a man-made polymerized nucleic acid.

Examples of clinically relevant single nucleotide polymorphisms in human genes are listed in Table 1 (available at www.sciencemag.org/feature/data/1044449.shl).

TABLE 1
Examples of clinically relevant genetic polymorphisms in human drug-
metabolizing enzymes, transporters, targets and receptors
Drug-metabolizing enzymes and transporters

| | | Consequences |
|---|---|---|
| Enzyme | Substrate | of polymorphism |
| CYP2A6 | Coumarin, nicotine, halothane | Cigarette |
| | | addiction |
| CYP2C9 | Tolbutamide, warfarin, | Anticoagulant |
| | phenytoin, nonsteroidal anti- | effect of warfarin |
| | inflammatories | |
| CYP2C19 | Mephenytoin, omeprazole, | Peptic ulcer cure |
| | hexobarbital, mephobarbital, | rates with |
| | propranolol, proguanil, | omeprazole |
| | phenytoin | |
| CYP2D6 | Beta blockers, antidepressants, | Tardive dyskin- |
| | antipsychotics, codeine, | esia from antipsy- |
| | debrisoquin, dextromethorphan, | chotics, narcotic |
| | encainide, flecainide, guanoxan, | side effects, effi- |
| | methoxyamphetamine, N- | cacy, and depend- |
| | propylajmaline, perhexiline, | ence, imipramine |
| | phenacetin, phenformin, | dose requirement, |
| | propafenone, sparteine | beta blocker |
| | | effect |
| CYP2E1 | N-Nitrosodimethylamine, | Possible effect on |
| | acetaminophen, ethanol | alcohol con- |
| | | sumption |
| Aldehyde | Cyclophosphamide, vinyl | SCE frequency in |
| dehydrogenase | chloride | lymphocytes |
| (ALDH2) | | |
| Alcohol | Ethanol | Increased alcohol |
| dehydrogenase | | consumption and |
| (ADH3) | | dependence |
| Dihydropyrimidine | Fluorouracil | 5-fluorouracil |
| dehydrogenase | | neurotoxicity |
| NQO1 (DT- | Ubiquinones, menadione, | Menadione-asso- |
| diaphorase) | mitomycin C | ciated urolithiasis |
| N-Acetyltransferase | Isoniazid, hydralazine, | Hypersensitivity |
| (NAT2) | sulfonamides, amonafide, | to sulfonamides, |
| | procainamide, dapsone, caffeine | amonafide toxi- |
| | | city, hydralazine- |
| | | induced lupus, |
| | | isoniazid neuro- |
| | | toxicity |
| Catechol-O- | Estrogens, levodopa, ascorbic | Substance abuse, |
| methyltransferase | acid | levodopa |
| | | response |
| Thiopurine | Mercaptopurine, thioguanine, | Thiopurine toxi- |
| methyltransferase | azathioprine | city and efficacy, |
| | | risk of second |
| | | cancers |
| UDP-glucuronosyl- | Irinotecan, bilirubin | Irinotecan |
| transferase | | glucuronidation |
| (UGT1A1) | | |

Drug receptors and targets

| | | Drug effects |
|---|---|---|
| Receptor/target | Medication | linked to poly- |
| | | morphisms |
| β2-Adrenergic | Albuterol | Response in |
| receptor | | asthmatics |
| 5-Lipoxygenase | ABT-761 (zileuton) | Response in |
| promoter | | asthmatics |
| Angiotensin- | Enalapril, isinopril, captopril | Renoprotective |
| converting enzyme | | effects, cardiac |
| (ACE) | | indices, blood |
| | | pressure, IgA |
| | | nephropathy |
| Cholesteryl ester | Pravastatin | Progression of |
| transfer protein | | atherosclerosis |
| Stromelysin | Pravastatin | Efficacy in coro- |
| | | nary atherosclero- |
| | | sis and restenosis |
| Angiotensin-II T | Perindopril nitrendipine | Change in arterial |
| receptor | | stiffness |
| Sulfonylurea | Tolbutamide | Serum C-peptide |
| receptor | | and insulin |
| | | response |
| 5-Hydroxytrypt- | Clozapine | Response in |
| amine 2C receptor | | schizophrenia |
| 5-Hydroxytrypt- | Clozapine and other | Response in |
| amine 2A receptor | neuroleptics | schizophrenia |
| Serotonin | Fluvoxamine | Response in de- |
| transporter | | lusional de- |
| promoter | | pression |
| Dopamine D2 and | Dopamine D2 and D3 receptors | Drug-induced |
| D3 receptors | | tardive dyskinesia |
| Vitamin D receptor | 1,25-Dihydroxy vitamin D3 | Vitamin D re- |
| | | sponse in rickets |
| Glucocorticoid | Dexamethasone | Cortisol and in- |
| receptor | | sulin response |
| Nicotinic receptor | Acetylcholine (-) nicotine | Increased sensi- |
| | | tivity to agonist |
| | | effects |
| Delta opioid | Heroin | Addiction |
| receptor | | |
| HERG | Quinidine, cisapride | Drug-induced |
| | | long QT syn- |
| | | drome, Drug- |
| | | induced torsade |
| | | de pointes |
| KvLQT1 | Terfenadine, disopyramide | Drug-induced |
| | meflaquine | long QT syn- |
| | | drome |
| hHCNE2 | Clarithromycin | Drug-induced |
| | | arrhythmia |
| SCN5A | Mexiletine | Response of |
| | | manic depressive |
| | | illness |
| Inositol-p1p | Lithium | Response of |
| | | manic depressive |
| | | illness |
| HLA-DRB1 | Cyclosporin A | Response of |
| | | aplastic anemia |
| Apolipoprotein E4 | Tacrine | Response of Alz- |
| | | heimer's disease |
| Ryanodine receptor | Halothane or succinylcholine | Drug-induced |
| | | malignant hyper- |
| | | thermia |
| Prothrombin | Oral contraceptives | Risk of cerebral- |
| | | vein thrombosis |
| Peroxisome | Insulin | Insulin sensitivity |
| proliferator- | | |
| activated receptor | | |

In addition, the present invention provides for analysis of deletion and insertion mutations. The following examples are provided. Human α-1-antitrypsin mRNA (Genbank Accession No. M11465), which has a 1 base pair deletion. The present invention takes advantage of the missing base as follows:

| | | |
|---|---|---|
| | wild-type allele | |
| 531 | CACGAAGAGG CCAAGAAACA GATCAACGAT TACGTGGAGA AGGGTACTCA | (SEQ ID:NO. 18) |
| | \| \| | |
| | 28 base product | |

A primer corresponding bases 535 to 557 is extended with dGTP, dATP and dTTP generates a 28 base pair product. A null granite mutation is missing the C at position 563 in the wild-type gene.

| | | |
|---|---|---|
| | mutant allele | |
| 531 | CACGAAGAGG CCAAGAAACA GATCAACGAT TAGTGGAGAA GGGTACTCA | (SEQ ID:NO. 19) |
| | \| \| | |
| | 41 base product | |

However, using same primer as described above, and the same dNTPs (dCTP, dATP, and dTTP), a 41 bp product is generated in the mutant allele. In this example, two products could be generated that are easily distinguishable from the 23 bp primer, and from each other (28 bp versus 41 bp).

The human triglyceride lipase gene, exon 3, (Genbank Accession No. M29188) is shown below with divergent sequences in bold:

| | | |
|---|---|---|
| | wild-type allele | |
| 171 | ccctggccca cgaccactac accatcgccg tccgcaacac ccgccttgtg ggcaaggagg | (SEQ ID:NO. 20) |
| | \| \| | |
| | 35 base product | |

A primer spanning bases 181 to 201 extended with dGTP, dCTP and dATP (thus omitting dTTP) generates a 35 bp fragment. Mutated human triglyceride lipase gene, exon 3 that contains an 18 bp insert (Genbank Accession No. AF037404):

| | | |
|---|---|---|
| | mutant allele | |
| 125 | ccctggccca cgaccactac accatcgccg tccactacac cgtcgccgtc cgcaac | (SEQ ID:NO. 21) |
| | \| \| | |
| | 25 base product | |

A primer spanning bases 135 to 155 (same sequence as described above) extended with dGTP, dCTP and dATP generates a 25 bp fragment. This is an example of an insertion that can be identified using this approach, since the 21 bp primer is easily distinguished from the both of the extension products at 35 bp and 25 bp, which are also easily distinguished from one another. For this, as for most SNPs, there are many options for application of the invention. Only one such example of these is presented here.

Deletion polymorphisms may also be identified by the methods, as in the following example. The mutant α-2-macroglobulin (A2M2-2) gene has a 5 bp deletion (divergent sequence is shown in bold) when compared to the wild-type A2M allele (Genbank Accession Nos. AF349032 and AF349033):

| | | |
|---|---|---|
| | wild-type allele | |
| 58 | atctgtat gtttattgta atgtcttctt cctcactcac catagagtca | SEQ ID:NO. 22) |
| | \| \| | |
| | 42 base product | |

A primer spanning bases 58-78 and extended with dTTP, dCTP and dATP will generate a 42 bp product.

| | | |
|---|---|---|
| | mutant allele | |
| 58 | atctgtat gtttattgta atgtcttctt cctcactcag agtcagatgta | SEQ ID:NO. 23) |
| | \| \| | |
| | 37 base product | |

However, for the mutant allele, a primer spanning bases 58-78 (same as above) and extended with dTTP, dCTP and dATP will generate a 37 bp product. The 32 bp and 37 bp extension products of the primer can be used to distinguish the primer and the extension products form one another, thereby identifying the wild-type and mutant alleles.

A portion of the wild-type beta-globulin gene (Genbank Accession No. L26463) is shown below and provides an opportunity to illustrate the detection of a deletion:

| | | |
|---|---|---|
| | wild-type allele | |
| 1075 | cccaccctta ggctgctggt ggtctaccct tggacccaga ggttctt | SEQ ID:NO. 24) |
| | \| \| | |
| | 32 base product | |

A primer spanning bases 1085 to 1105 extended with dGTP, dCTP and dATP will generate a 32 base pair product. The mutant beta globulin gene (Genbank Accession No. U20223) is shown below (the 2 base deletion is indicated by dashes):

| | | |
|---|---|---|
| | mutant allele | |
| 391 | cccaccctta ggctgctggt ggtctaccct tgga--caga ggttctt | (SEQ ID:NO. 25) |
| | \| \| | |
| | 30 base product | |

A primer spanning bases 401 to 421 (same sequence as above) extended with dGTP, dCTP and dATP will generate a 30 base pair product. The 30 bp and 32 bp extension products are easily distinguishable and are easily separated from the 21 bp primer.

These four examples illustrate the broad applicability and flexibility of the present invention as applied to a variety of different mutations.

II. Amplifying the Target Sequence

In a particular embodiment, it may be desirable to amplify the target sequence before evaluating the polymorphism. Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA. The DNA also may be from a cloned source or synthesized in vitro.

The term “primer,” as used herein, is meant to encompass any nucleic acid that is capable of priming the synthesis of a nascent nucleic acid in a template-dependent process. Typically, primers are oligonucleotides from ten to twenty or thirty base pairs in length, but longer sequences can be employed. Primers may be provided in double-stranded or single-stranded form, although the single-stranded form is preferred.

Pairs of primers designed to selectively hybridize to nucleic acids flanking the polymorphic site are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

It is also possible that multiple target sequences will be amplified in a single reaction. Primers designed to expand specific sequences located in different regions of the target genome, thereby identifying different polymorphisms, would be mixed together in a single reaction mixture. The resulting amplification mixture would contain multiple amplified regions, and could be used as the source template for polymorphism detection using the methods described in this application.

A number of template dependent processes are available to amplify the oligonucleotide sequences present in a given template sample. One of the best known amplification methods is the polymerase chain reaction (referred to as PCR™), which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed when the source of nucleic acid is fractionated or whole cell RNA. Methods of reverse transcribing RNA into cDNA are well known (see Sambrook et al., 2001). Alternative methods for reverse polymerization utilize thermostable DNA polymerases. These methods are described in WO 90/07641. Polymerase chain reaction methodologies are well known in the art. Representative methods of RT-PCR are described in U.S. Pat. No. 5,882,864.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Another ligase-mediated reaction is disclosed by Guilfoyle et al. (1997). Genomic DNA is digested with a restriction enzyme and universal linkers are then ligated onto the restriction fragments. Primers to the universal linker sequence are then used in PCR to amplify the restriction fragments. By varying the conditions of the PCR, one can specifically amplify fragments of a certain size (i.e., less than a 1000 bases). An example for use with the present invention would be to digest genomic DNA with BamHI, and ligate on M13-universal primers with an BamHI over hang, followed by amplification of the genomic DNA with an M13 universal primer. Only a small percentage of the total DNA would be amplified (the restriction fragments that were less than 1000 bases). One would then use labeled primers that correspond to a SNP that are located within XbaI restriction fragments of a certain size (<1000 bases) to perform the assay. The benefit to using this approach is that each individual region would not have to be amplified separately. There would be the potential to screen thousands of SNPs from the single PCR reaction, i.e., multiplex potential.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence, which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include polymerization-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 discloses a nucleic acid amplification process involving cyclically synthesizing single-stranded RNA (ssRNA), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (ssDNA) followed by polymerization of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

Another advantageous step is to prevent unincorporated NTPs from being incorporated in a subsequent primer extension reaction. Commercially available kits may be used to remove unincorporated NTPs from the amplification products. The use of shrimp alkaline phosphatase to destroy unincorporated NTPs is also a well-known strategy for this purpose.

III. Evaluating the Polymorphism

A. Selection of Oligonucleotide Primers and dNTPs

The present invention requires an oligonucleotide primer that is selected to bind the DNA sequence more than one nucleotide upstream of the target polymorphism. The invention requires the use of only one primer, which may be in either the forward or reverse orientation. Due to the fact that no more than three NTPs are present in the extension reaction, the success of the invention depends on selecting a primer that hybridizes to the target DNA such that the region between the 3′ end of the primer and the target polymorphism does not contain the omitted NTP.

Various combinations of no more than three NTPs may be employed in the extension reaction. By way of illustration, where dATP is “A,” dCTP is “C,” dGTP is “G,” and dTTP is “T” (rUTP is U), the following permutations are exemplary (where U may be substituted for T):

A/T/G A/C/G A/G C/T C/G G A
C/T/G A/C/T A/T G/T A/C T C

Various other combinations using alternative NTPs are contemplated, including inosine, NTP derivatives, and iodinated, or bromylated NTPs.

In designing a primer, it may also be desirable to select the length of the primer such that the extension products will be separable within a desired size range. For example, an investigator might choose to add 15 bases of irrelevant sequence to the primer in order that the primer and extension products would be easily differentiated by size from a primer with no such addition of irrelevant sequences. It is possible to identify multiple polymorphisms in a single reaction, although identifying suitable probe/polymorphism combination for multiple sites using the same polymerization cocktail is much more complicated than when a single site is selected. It is also possible to perform multiple polymerase chain reactions at once.

A computer program has been written to design primers for the initial amplification of the target region and the primer used for the assay. The program titled “KSAPP” is written in the Perl programming language. This program utilizes DNA sequences, in the preferred embodiment, downloaded from the NCBI Genbank database. Two input files are required: the sequence file saved in a FASTA sequence file format, and the description of the sequence saved in the Genbank file format. The software program uses the Genbank file to identify the location of the polymorphisms in the FASTA file, as well as the type of polymorphism. The program then examines the sequence flanking the polymorphic site to design primers that could amplify the region of interest. Users of the computer program can input parameters that will limit the size of the amplified region as well as the composition of the primers (such as percentage of dGTP/dCTP to dATP/dTTP). The computer program then determines if a primer can be designed on either the coding or non-coding strand that can be used for the actual assay, and which base or bases will be the omitted from the assay reaction. The computer program then produces an output file for each polymorphism meeting the criteria to be used in this assay. The output file contains the identification of the polymorphism (using the unique RS number from the National Center for Biotechnology Information (NCBI) SNP database), the position of the polymorphism in the sequence, the type of polymorphism, the primers to amplify the region, the size of the amplified region, the assay primer, the dNTP omitted from the assay reaction, and the sizes of the different alleles at the polymorphism.

B. Oligonucleotide Synthesis

Oligonucleotide synthesis is well known to those of skill in the art. Various mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference in its entirety. Basically, chemical synthesis can be achieved by the diester method, the triester method, and the polynucleotides phosphorylase method and by solid-phase chemistry. These methods are discussed in further detail below.

Diester method. The diester method was the first to be developed to a usable state, primarily by Khorana and co-workers (Khorana, 1979). The basic step is the joining of two suitably protected deoxynucleotides to form a dideoxynucleotide containing a phosphodiester bond. The diester method is well established and has been used to synthesize DNA molecules (Khorana, 1979).

Triester method. The main difference between the diester and triester methods is the presence in the latter of an extra protecting group on the phosphate atoms of the reactants and products (Itakura et al., 1975). The phosphate protecting group is usually a chlorophenyl group, which renders the nucleotides and polynucleotide intermediates soluble in organic solvents. Therefore, purifications are done in chloroform solutions. Other improvements in the method include (i) the block coupling of trimers and larger oligomers, (ii) the extensive use of high-performance liquid chromatography for the purification of both intermediate and final products, and (iii) solid-phase synthesis.

Polynucleotide phosphorylase method. This is an enzymatic method of DNA synthesis that can be used to synthesize many useful oligodeoxynucleotides (Gillam et al., 1978). Under controlled conditions, polynucleotide phosphorylase adds predominantly a single nucleotide to a short oligodeoxynucleotide. Chromatographic purification allows the desired single adduct to be obtained. At least a trimer is required to initiate the method of adding one base at a time, a primer that must be obtained by some other method. The polynucleotide phosphorylase method works and has the advantage that the procedures involved are familiar to most biochemists.

Solid-phase methods. The technology developed for the solid-phase synthesis of polypeptides has been applied to nucleic acids. It has been possible to attach the initial nucleotide to solid support material followed by proceeding with the stepwise addition of nucleotides. All mixing and washing steps are simplified, and the procedure becomes amenable to automation. These syntheses are now routinely carried out using automatic DNA synthesizers.

Phosphoramidite chemistry (Beaucage, 1993) has become by far the most widely used coupling chemistry for the synthesis of oligonucleotides. As is well known to those skilled in the art, phosphoramidite synthesis of oligonucleotides involves activation of nucleoside phosphoramidite monomer precursors by reaction with an activating agent to form activated intermediates, followed by sequential addition of the activated intermediates to the growing oligonucleotide chain (generally anchored at one end to a suitable solid support) to form the oligonucleotide product.

C. Primer Extension

The primer and no more than three NTPs are combined with a polymerase and the target sequence, which serves as a template for amplification. As described above, by using less than all four NTPs, it is possible to omit one or more of the polymorphic nucleotides needed for incorporation at the polymorphic site. It is important for the practice of the present invention that the amplification be designed such that the omitted nucleotide(s) is(are) not required between the 3′ end of the primer and the target polymorphism. The primer is then extended by a nucleic acid polymerase, in a preferred embodiment by Taq polymerase. If the omitted NTP is required at the polymorphic site, the primer is extended up to the polymorphic site, at which point the polymerization ceases. However, if the omitted NTP is not required at the polymorphic site, the primer will be extended beyond the polymorphic site, creating a longer product. Detection of the extension products is based on, for example, separation by size/length which will thereby reveal which polymorphism is present.

D. Separation and Detection of Nucleic Acids

Following the amplification, it may be desirable to assess the length of the amplification product or products. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the skilled artisan may remove the separated band by heating the gel, followed by extraction of the nucleic acid.

Separation of nucleic acids may also be effected by chromatographic techniques known in the art. There are many kinds of chromatography that may be used in the practice of the present invention, including capillary adsorption, partition, ion-exchange, hydroxylapatite, molecular sieve, reverse-phase, column, paper, thin-layer, and gas chromatography as well as HPLC.

Amplification products may be visualized in order to confirm amplification of the target sequences. In one embodiment, the primer is conjugated to a chromophore but may instead be radiolabeled or fluorometrically labeled. In another embodiment, the primer is conjugated to a binding partner that carries a detectable moiety, such as an antibody or biotin. In other embodiments, the primer incorporates a fluorescent dye or label. In yet other embodiments, the primer has a mass label that can be used to detect the molecule amplified. Other embodiments also contemplate the use of Taqman™ and Molecular Beacon™ probes. Alternatively, one or more of the dNTPs may be labeled with a radioisotope, a fluorophore, a chromophore, a dye or an enzyme. Also, chemicals whose properties change in the presence of DNA can be used for detection purposes. For example, the methods may involve staining of a gel with, or incorporation into the separation media, a fluorescent dye, such as ethidium bromide or Vistra Green, and visualization under an appropriate light source.

Another method well suited for the detection of small DNA molecules is mass spectrometry (MS). Of the various mass spectrometry applications available, Matrix-Assisted Laser Desorption/Ionization-Time Of Flight (MALDI-TOF) is most the commonly employed for examining nucleic acids. MALDI-TOF is suitable for high-throughput industrial settings and can resolve the size of relatively short oligonucleotides. While small compared to nucleic acids from biologic sources, oligonucleotides are nevertheless relatively large molecules, ranging in size from about 6000 daltons for a 20-mer to 30,000 daltons for a 100-mer. MALDI-TOF is useful for compounds up to ˜15,000 daltons, or about a 50-mer oligonucleotide. A particular product is Sequenom's MassARRAY™ platform for high-throughput mass spec analysis of oligonucleotides, which provides high-speed Bruker MALDI-TOF instruments integrated with proprietary mass analysis software. Samples >15,000 daltons do not ionize/fly effectively and are thus outside the optimal resolution range of MALDI-TOF. Electrospray Ionization-Time of Flight (ESI-TOF) MS can resolve longer oligonucleotides (up to ˜80 bases in length), but this platform is more salt-sensitive, slower, and less suitable for high throughput applications.

In MALDI-TOF, oligonucleotides are mixed with a carrier, usually, picolinic acid, and are deposited as a matrix on a grid. The instrument pulses laser light to vaporize the matrix in a process known as “desorption.” Some molecules become ionized through protonation (i.e., the gain of a proton). In time of flight (TOF) mass spectrometry, ionized molecules are accelerated by an electrostatic field in the mass analyzer to a common kinetic energy. With the same kinetic energy, lighter ions travel faster and heavier ions travel more slowly. The ionized particles enter at one end of the TOF tube, and the number of ions reaching a detector at the other end is recorded in a time-dependent manner. The lightest ions reach the detector first and the heaviest arrive last. The entire mass spectrum is recorded and the information sorted by computer.

Alternatively, visualization may be achieved indirectly. Following separation of the amplification products, a labeled nucleic acid probe is hybridized with the amplification products. The probe preferably is conjugated to a chromophore but may be radiolabeled. In another embodiment, the probe is conjugated to a binding partner, such as an antibody or biotin, or another binding partner carrying a detectable moiety. In other embodiments, the probe incorporates a fluorescent dye or label. In yet other embodiments, the probe has a mass label that can be used to detect the molecule amplified. Other embodiments also contemplate the use of Taqman™ and Molecular Beacon™ probes. In still other embodiments, solid-phase capture methods combined with a standard probe may be used as well.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference in its entirety.

The choice of label incorporated into the extension products is dictated by the method used for analysis. When using capillary electrophoresis, microfluidic electrophoresis, HPLC, or LC separations, either incorporated or intercalated fluorescent dyes are used to label and detect the amplification products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, the primer for the extension reaction can be labeled with a fluorophore, a chromophore or a radioisotope, or by associated enzymatic reaction. Alternatively, if polyacrylamide gel or slab gel electrophoresis is used, one or more of the NTPs in the extension reaction can be labeled with a fluorophore, a chromophore or a radioisotope, or by associated enzymatic reaction. Enzymatic detection involves binding an enzyme to a nucleic acid, e.g., via a biotin:avidin interaction, following separation of the amplification products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If the extension products are separated using a mass spectrometer no label is required because nucleic acids are detected directly.

In the case of radioactive isotopes, tritium, ¹⁴C and ³²P are used predominantly. Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

A number of the above separation platforms can be coupled to achieve separations based on two different properties. For example, some of the primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

E. Genotyping

In one context, the present invention may be used to detect alleles present in a polynucleotide sample (either DNA or RNA). If the target polynucleotide is haploid, then a single product will be generated that is determined by the single allele present in the sample. If the target polynucleotide is diploid, then more than one product may be generated. If the sample homozygous for one of the alleles, then a single product will be generated. If the sample is heterozygous for the polymorphism being detected, then two products will be observed.

Another embodiment of this application is to pool various samples and determine the amount of each genotypic product in the pooled sample. This could be used to determine the percentage of each allele in the pooled sample. For example, if the assay products are measured with flourescence, then the amount of flourescence for each product could be determined. The ratio of the flourescence signals would represent the relative allele frequency of the alleles in the samples being tested.

VI. Kits

All the essential materials and reagents required for detecting nucleic acid mutations in a sample may be assembled together in a kit. This generally will comprise a primer designed to hybridize specifically to target nucleotides at the appropriate position upstream of the polymorphism of interest. The primer may be labeled with a radioisotope, a fluorophore, a chromophore, a dye, an enzyme, or TOF carrier. Also included may be enzymes suitable for amplifying nucleic acids, including various polymerases (reverse transcriptase, Taq, etc.), dNTPs/rNTPs and buffers (e.g., 10×buffer=100 mM Tris-HCl (pH 8.3), and 500 mM KCl) to provide the necessary reaction mixture for amplification. One or more of the deoxynucleotides may be labeled with a radioisotope, a fluorophore, a chromophore, a dye, or an enzyme. Such kits may also include enzymes and other reagents suitable for detection of specific nucleic acids or amplification products.

The container means of the kits will generally include at least one vial, test tube, flask, bottle, or other container means, into which a component may be placed, and preferably, suitably aliquoted. Where there is more than one component in the kit, the kit also will generally contain additional containers into which the additional components may be separately placed. However, various combinations of components may be comprised in a container. The kits of the present invention also will typically include a means for packaging the component containers in close confinement for commercial sale. Such packaging may include injection or blow-molded plastic containers into which the desired component containers are retained.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Detection of a Single Base Polymorphism in FcγRIIA (Method I)

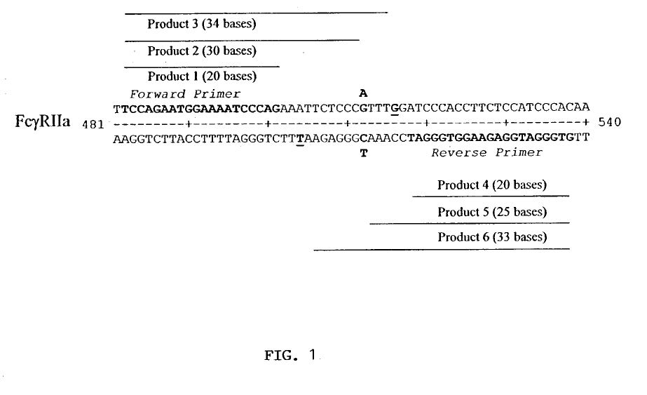

The human FcγRIIA gene is polymorphic at the nucleic acid position that encodes amino acid 131 (Genbank Accession Code: M90724). This amino acid may be histadine or arginine as a result of an adenine or a guanine at the critical position of nucleotide 512 (FIG. 1). This difference changes the affinity of FcγRIIa for some of the subclasses of IgG and has been associated with systemic lupus erythematosus, especially with lupus nephritis (Salmon et al., 1996), among many other consequences.

A primer is prepared that is labeled with the IRDye™700 (700 nm dye) (Li-Cor Part number 4200-33, M.F. C₅₂,H₆₇N₄O₅PS) (molecular weight is 981.15; absorption maximum is at 681 nm; emission maximum is at 712 nm; molar absorption is 170,000; and quantum efficiency is 47.7% in methanol). For example, the primer could be covalently bound to the dye: (700 nm dye) TCCAGCCTGGAAAATCCCAG.

When dGTP is omitted from the primer extension reaction the final product for the “C” allele is (700 nm dye)-TCCAGCCTGGAAAATCCCAG-AAATTCTCCC. In this situation the dNTPs included in the reaction are dATP, dCTP, and dTTP. The final product of the “T” allele is (700 nm dye)-TCCAGCCTGGAAAATCCCAG-AAATTCTCCC-AAACCTA. The dashes shonw are included to emphasize the covalent binding of the dye, and to illustrate the three different sizes of the products expected.

Alternatively, the primer could be prepared for the other strand of DNA (the reverse complement) and labeled, for example, with the IRDye™800 (800 nm dye) (Li-Cor Part number 4000-33, M.F. C₅₉,H₇₅N₄O₆PS) (molecular weight is 999.3; absorption maximum is at 787 nm; emission maximum is at 812 nm; molar absorption is 275,000; and quantum efficiency is 15% in methanol).

Here the labeled primer could be this (800 nm dye)-TGGGATGGAGAAGTGGGAT-. When dTTP is omitted from the reaction the final product for the “T” allele is (800 nm dye)-TGGGATGGAGAAGTGGGAT-CCAAA and for the “C” allele is (800 nm dye)-TGGGATGGAGAAGTGGGA-CCAAA-GGGAGAA-. In this situation the dNTPs included in the reaction are dATP, dCTP, and dGTP.

The sequence of this region of FcγRIIA in FIG. 1 illustrates the primers based upon the forward and reverse sequences.

Method I was performed as described above using the forward primer (FIG. 1) to detect the polymorphisms encoding H131R of FcγRIIA. Twenty-one samples, from subjects whose genotype was previously determined by sequencing their DNA, were tested. For each sample polymerase chain reaction (PCR™) was setup using 1.6 μL genomic DNA (0.01 μg/μL), 6.5 μL autoclaved H₂O, 1 μL of 10×PCR buffer, 0.6 μL of 25 mM MgCl₂, 1 μL of 4×2 mM dNTP (including all four dNTPs: dATP, dTTP, dCTP, and dGTP), 0.05 μL forward primer (20 μM), 0.05 μL of reverse primer (20 μM), and 0.075 μL Taq polymerase (of 5 units/μl). Reactions were heated to 95° C. for 5 minutes. Then 14 cycles were performed at 94° C. for 20 seconds, 58° C. for 1 minute, and 72° C. for 30 seconds. Then 28 cycles were performed of 94° C. for 20 seconds, 55° C. for 1 minute, and 72° C. for 30 seconds. The last step is a 10 minute extension at 72° C. for 10 minutes. The samples are then held at 4° C. PCR reactions were purified using Milli-Pore PCR™ clean up kits following the manufacturer's instructions. Treatment of PCR™ reactions with shrimp alkaline phosphatase is a preferred embodiment that is nearly perfect in the inventors' experiments.

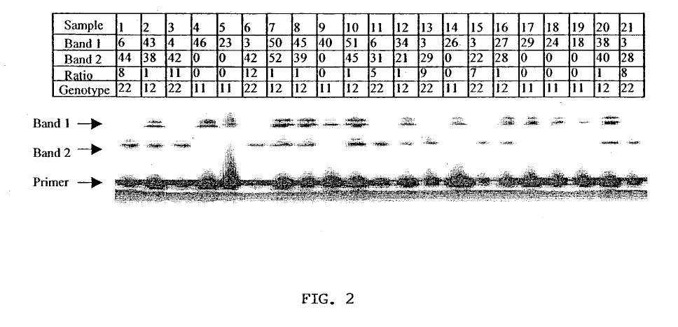

SNP genotyping reactions were setup on each sample using 3.475 μL of autoclaved H₂O, 0.6 μL of purified PCR™ product, 0.6 μL of 10×Buffer, 0.6 μL of 2.5 mm dATP, dTTP, and dCTP (−dGTP, minus dGTP or lacking dGTP), 0.075 μL Taq polymerase (of 5 units/μl), 0.4 μL of 25 mM MgCl₂, and 0.25 μL of 700 nm labeled primer. Reactions were heated to 95° C. for 2 minutes. Then 30 cycles were performed at 92° C. for 30 seconds, 45° C. for 15 seconds, and 72° C. for 15 seconds. Reactions were diluted 10-fold and evaluated on a Li-Cor 4200 automated DNA sequencer. Data were obtained from all 21 samples (FIG. 2). In FIG. 2, Band 1 corresponds to the “A” allele that encodes histidine at amino acid position 131 and Band 2 corresponds to “G” allele that encodes arginine at amino acid position 131. Intensities of the bands were measured using UVP Labworks version 4.0 software. Intensities were divided by 1000 and rounded to the nearest whole number. For each sample the ratio (Band 1/Band 2) was determined and rounded off to the nearest whole number. A ratio of 0 corresponded to the homozygous AA genotype, a ratio>1 corresponded to a the homozygous GG genotype, and a ratio equal to 1 corresponded to the heterozygous AG genotype. Ratios for band intensites were determined to overcome problems with background bands caused by contamination of dGTP from the original PCR™ reaction. All 21 samples were in perfect agreement with previous DNA sequencing results. In this preliminary evaluation the sensitivity of the method is 100%, the specificity is 100% and the predictive value is 100% in these samples.

In this embodiment of the invention, it is important that the unextended, labeled primer be at least two and preferably more than two nucleotides shorter than the product made by extension reaction for either allele. Similarly, the method is most successful when the sequence allows the two (or more) alleles to be separated by two or more nucleotide bases, though one base differences may also be detected.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope, and concept of the invention as defined by the appended claims.

VIII. References

The following references, to the extent that they provide exemplary procedural or other details supplementary to those set forth herein, are specifically incorporated herein by reference.

U.S. Pat. No. 4,659,774

U.S. Pat. No. 4,683,195

U.S. Pat. No. 4,683,202

U.S. Pat. No. 4,800,159

U.S. Pat. No. 4,816,571

U.S. Pat. No. 4,883,750

U.S. Pat. No. 4,959,463

U.S. Pat. No. 5,141,813

U.S. Pat. No. 5,264,566

U.S. Pat. No. 5,428,148

U.S. Pat. No. 5,554,744

U.S. Pat. No. 5,574,146

U.S. Pat. No. 5,602,244

U.S. Pat. No. 5,840,873

U.S. Pat. No. 5,843,640

U.S. Pat. No. 5,843,650

U.S. Pat. No. 5,843,651

U.S. Pat. No. 5,846,708

U.S. Pat. No. 5,846,709

U.S. Pat. No. 5,846,717

U.S. Pat. No. 5,846,726

U.S. Pat. No. 5,846,729

U.S. Pat. No. 5,846,783

U.S. Pat. No. 5,849,487

U.S. Pat. No. 5,849,497

U.S. Pat. No. 5,849,546

U.S. Pat. No. 5,849,547

U.S. Pat. No. 5,853,990

U.S. Pat. No. 5,853,992

U.S. Pat. No. 5,853,993

U.S. Pat. No. 5,856,092

U.S. Pat. No. 5,858,652

U.S. Pat. No. 5,861,244

U.S. Pat. No. 5,863,732

U.S. Pat. No. 5,863,753

U.S. Pat. No. 5,866,331

U.S. Pat. No. 5,866,366

U.S. Pat. No. 5,882,864

U.S. Pat. No. 5,905,024

U.S. Pat. No. 5,910,407

U.S. Pat. No. 5,912,124

U.S. Pat. No. 5,912,145

U.S. Pat. No. 5,912,148

U.S. Pat. No. 5,916,776

U.S. Pat. No. 5,916,779

U.S. Pat. No. 5,919,630

U.S. Pat. No. 5,922,574

U.S. Pat. No. 5,925,517

U.S. Pat. No. 5,928,862

U.S. Pat. No. 5,928,869

U.S. Pat. No. 5,928,905

U.S. Pat. No. 5,928,906

U.S. Pat. No. 5,929,227

U.S. Pat. No. 5,932,413

U.S. Pat. No. 5,932,451

U.S. Pat. No. 5,935,791

U.S. Pat. No. 5,935,825

U.S. Pat. No. 5,939,291

U.S. Pat. No. 5,942,391

Beaucage, Methods Mol. Biol., 20:33-61,1993.

European Application No. 320 308

European Application No. 329 822

Frohman, In: PCR Protocols: A Guide To Methods And Applications, Academic Press, N.Y., 1990.

GB Application No. 2 202 328

Gillam et al., J. Biol. Chem., 253(8):2532-2539, 1978.

Guilfoyle et al., Nucleic Acids Research, 25:1854-1858, 1997.

Innis et al., Proc. Natl. Acad. Sci. USA, 85(24):9436-9440, 1988.

Itakura et al., J. Am. Chem. Soc., 97(25):7327-7332, 1975.

Khorana, Science, 203(4381):614-625, 1979.

Kwoh et al., Proc. Natl. Acad. Sci. USA, 86: 1173, 1989.

Ohara et al., Proc. Natl. Acad. Sci. USA, 86: 5673-5677, 1989.

PCT Application No. PCT/US87/00880

PCT Application No. PCT/US89/01025

PCT Application No. WO 88/10315

PCT Application No. WO 89/06700

Salmon et al., J. Clin. Invest., 97(5):1348-1354, 1996.

Sambrook et al., In: Molecular cloning, Cold Spring Harbor Laboratory Press, Cold Spring Harbor, N.Y., 2001.

Walker et al., Proc. Natl. Acad. Sci. USA, 89:392-396 1992.

What is claimed is:

1. A method for identifying a nucleic acid polymorphism comprising:
(a) providing a target polynucleotide comprising a first polymorphic site;
(b) providing a first oligonucleotide that hybridizes upstream of the first polymorphic site, the space between the ‘3 end of the first oligonucleotide and the first polymorphic site defined as a first intervening region, wherein the first intervening region lacks one of the bases that occurs at the first polymorphic site;
(c) mixing the first oligonucleotide and the polynucleotide with a polymerase and a polymerization cocktail of no more than three different NTPs, wherein the cocktail lacks an NTP for the same base that is missing from the intervening region;
(d) subjecting the mixture to conditions that support polymerization; and
(e) assessing the length of polymerization products from step (d),
wherein a polymerization product equal in size to the first oligonucleotide plus the first intervening region indicates that polymerization terminated at the polymorphic site, and that the first polymorphic site contains the base from the NTP lacking from the polymerization cocktail, and wherein a polymerization product greater in size than the first oligonucleotide plus the first intervening region indicates that polymerization continued past the first polymorphic site, and that the first polymorphic site contains a base from an NTP present in the polymerization cocktail.

2. The method of claim 1, wherein the oligonucleotide is labeled.

3. The method of claim 2, wherein the label is a radioisotope, a fluorophore, a chromophore, a dye, an enzyme, or a spectroscopic carrier.

4. The method of claim 1, wherein step (b) further comprises providing a second oligonucleotide that hybridizes upstream of a second polymorphic site, the space between the ‘3 end of the second oligonucleotide and the second polymorphic site defined as the second intervening region; wherein the second intervening region lacks one of the bases that occurs at the first and second polymorphic sites,
wherein a polymerization product equal in size to the second oligonucleotide plus the second intervening region indicates that polymerization terminated at the second polymorphic site, and that the second polymorphic site contains the base from the NTP lacking from the polymerization cocktail, and wherein a polymerization product greater in size than the second oligonucleotide plus the second intervening region indicates that polymerization continued past the second polymorphic site, and that the second polymorphic site contains a base from an NTP present in the polymerization cocktail.

5. The method of claim 4, wherein the second oligonucleotide plus second intervening region varies in size from the first oliognonucleotide plus first intervening region.

6. The method of claim 4, wherein the second oligonucleotide is labeled such that the polymerization product generated by the second oligonucleotide can be distinguished from the polymerization product generated by the first oliognonucleotide.

7. The method of claim 1, wherein the first oligonucleotide is about 10 to about 10,000 bases in length.

8. The method of claim 1, wherein the first oligonucleotide is about 10 to about 100 bases in length.

9. The method of claim 1, wherein the first oligonucleotide is 10 to about 25 bases in length.

10. The method of claim 1, wherein that portion of the first oliogonucleotide that hybridizes to the target polynucleotide is about 10 to about 80 bases.

11. The method of claim 1, wherein that portion of the first oliogonucleotide that hybridizes to the target polynucleotide is 10 to about 40 bases.

12. The method of claim 1, wherein that portion of the first oliogonucleotide that hybridizes to the target polynucleotide is 10 to 25 bases.

13. The method of claim 1, wherein assessing the length of the polymerization product is comprises electrophoretic separation.

14. The method of claim 1, wherein assessing the length of the polymerization product comprises chromatography.

15. The method of claim 13, wherein the separated polymerization product is stained with silver stain or intercalating dye.

16. The method of claim 14, wherein the separated polymerization product is stained with silver stain or intercalating dye.

17. The method of claim 1, wherein assessing the length of the polymerization product is determined by mass spectroscopy.

18. The method of claim 1, further comprising, prior to step (e) but following step (d), the additional steps of:
(i) dissociating polymerization products from the target polynucleotide sequence;
(ii) annealing second copy of the first oligonucleotide to the target polynucleotide;
(iii) mixing the second copy of the first oligonucleotide and the target polynucleotide with a polymerase and a cocktail of no more than three different NTPs, wherein the cocktail lacks a NTP for the same base that is missing from the intervening region; and
(iv) subjecting the mixture to conditions that support polymerization.

19. The method of claim 18, wherein steps (i)-(iv) are repeated.

20. The method of claim 1, wherein the polymerase is a thermostable polymerase.

21. The method of claim 1, wherein the polymorphic site comprises a deletion.

22. The method of claim 1, wherein the polymorphic site comprises an insertion.

23. The method of claim 1, wherein the polymorphic site comprises an inversion.

24. The method of claim 1, wherein the polymorphic site comprises a single nucleotide polymorphism.

25. The method of claim 1, wherein the intervening sequence is 1-50 bases.

26. The method of claim 1, wherein the intervening sequence is 2-25 bases.

27. The method of claim 1, wherein the intervening sequence is 3-10 bases.

28. The method of claim 1, wherein the intervening sequence is 2, 3, 4, 5, 6, 7, 8, 9, or 10 bases.

29. The method of claim 1, wherein the cocktail comprises only two NTPs, and the intervening region contains only the bases corresponding to the two NTP's represented in the cocktail.

30. The method of claim 1, wherein the cocktail comprises only one NTP, and the intervening region contains only the base corresponding to the NTP represented in the cocktail.

31. A method for identifying a nucleic acid polymorphism comprising:
(a) providing a target polynucleotide sequence comprising a first polymorphic site;
(b) providing a first oligonucleotide that hybridizes upstream of the polymorphic site, the space between the 3′ end of the first oligonucleotide and the first polymorphic site defined as a first intervening region, wherein the first intervening region lacks one of the bases that occurs at the first polymorphic site;
(c) mixing the first oligonucleotide and the polynucleotide with a polymerase and a polymerization cocktail of no more than three different NTPs, at least one of which is labeled, wherein the cocktail lacks a NTP for the same base that is missing from the intervening region;
(d) subjecting the mixture to conditions that support polymerization; and
(e) assessing the length of polymerization products,
wherein a polymerization product equal in size to the first oligonucleotide plus the first intervening region indicates that polymerization terminated at the polymorphic site, and that the first polymorphic site contains the base from the NTP lacking from the polymerization cocktail, and wherein a first polymerization product greater in size than the first oligonucleotide plus the first intervening region indicates that polymerization continued past the first polymorphic site, and that the first polymorphic site contains a base from an NTP present in the polymerization cocktail.

32. The method of claim 31, wherein the labeled NTP is labeled with a radioisotope, a fluorophore, a chromophore, a dye or an enzyme.

33. The method of claim 31, wherein step (b) further comprises providing a second oligonucleotide that hybridizes upstream of a second polymorphic site, the space between the ‘3 end of the second oligonucleotide and the second polymorphic site defined as the second intervening region; wherein the second intervening region lacks one of the bases that occurs at the first and second polymorphic sites; wherein the second oligonucleotide plus second intervening region varies in size from the first oliognonucleotide plus first intervening region,
wherein a polymerization product equal in size to the oligonucleotide plus the second intervening region indicates that polymerization terminated at the second polymorphic site, and that the second polymorphic site contains the base from the NTP lacking from the polymerization cocktail, and wherein a polymerization product greater in size than the oligonucleotide plus the second intervening region indicates that polymerization continued past the second polymorphic site, and that the second polymorphic site contains a base from an NTP present in the polymerization cocktail.

34. The method of claim 31, wherein the first oligonucleotide is about 10 to about 10,000 bases in length.

35. The method of claim 31, wherein the first oligonucleotide is 10 to about 100 bases in length.

36. The method of claim 31, wherein the first oligonucleotide is 10 to about 25 bases in length.

37. The method of claim 31, wherein that portion of the first oliogonucleotide that hybridizes to the target polynucleotide is about 10 to about 80 bases.

38. The method of claim 31, wherein that portion of the first oliogonucleotide that hybridizes to the target polynucleotide is 10 to about 40 bases.

39. The method of claim 31, wherein that portion of the first oliogonucleotide that hybridizes to the target polynucleotide is 10 to 25 bases.

40. The method of claim 31, wherein assessing the length of polymerization products comprises electrophoretic separation.

41. The method of claim 31, wherein assessing the length of polymerization products comprises chromatography.

42. The method of claim 31, wherein assessing the length of polymerization products comprises mass spectroscopy.

43. The method of claim 31, further comprising, prior to step (e) but following step (d), the additional steps of:
(i) dissociating the polymerization products from the target polynucleotide sequence;
(ii) annealing second copy of the first oligonucleotide to the target polynucleotide;
(iii) mixing the second copy of the first oligonucleotide and the target polynucleotide with a polymerase and a polymerization cocktail of no more than three different NTPs, wherein the cocktail lacks a NTP for the same base that is missing from the intervening region; and
(iv) subjecting the mixture to conditions that support polymerization.

44. The method of claim 43, wherein steps (i)-(iv) are repeated.

45. The method of claim 31, wherein the polymerase is a thermostable polymerase.

46. The method of claim 31, wherein the polymorphic site comprises a deletion.

47. The method of claim 31, wherein the polymorphic site comprises an insertion.

48. The method of claim 31, wherein the polymorphic site comprises an inversion.

49. The method of claim 31, wherein the polymorphic site comprises a single nucleotide polymorphism.

50. The method of claim 31, wherein the intervening sequence is 1-50 bases.

51. The method of claim 31, wherein the intervening sequence is 2-25 bases.

52. The method of claim 31, wherein the intervening sequence is 3-10 bases.

53. The method of claim 31, wherein the intervening sequence is 2, 3, 4, 5, 6, 7, 8, 9, or 10 bases.


SEQUENCE LISTING (25 sequences)

SEQ ID NO: 1 - DNA, 20 bp, Artificial Sequence
  Description of Artificial Sequence  Synthetic Primer
tccagaatggaaaatcccag

SEQ ID NO: 2 - DNA, 10 bp, Artificial Sequence
  Description of Artificial Sequence  Synthetic Primer
aaattctccc

SEQ ID NO: 3 - DNA, 14 bp, Artificial Sequence
  Description of Artificial Sequence  Synthetic Primer
aaattctcccattt

SEQ ID NO: 4 - DNA, 21 bp, Artificial Sequence
  Description of Artificial Sequence  Synthetic Primer
gtgggatggagaaggtgggat

SEQ ID NO: 5 - DNA, 5 bp, Artificial Sequence
  Description of Artificial Sequence  Synthetic Primer
ccaaa

SEQ ID NO: 6 - DNA, 13 bp, Artificial Sequence
  Description of Artificial Sequence  Synthetic Primer
ccaaatgggagaa

SEQ ID NO: 7 - DNA, 20 bp, Artificial Sequence
  Description of Artificial Sequence  Synthetic Primer
tccagcctggaaaatcccag

SEQ ID NO: 8 - DNA, 30 bp, Artificial Sequence
  Description of Artificial Sequence  Synthetic Primer
tccagcctggaaaatcccagaaattctccc

SEQ ID NO: 9 - DNA, 37 bp, Artificial Sequence
  Description of Artificial Sequence  Synthetic Primer
tccagcctggaaaatcccagaaattctcccaaaccta

SEQ ID NO: 10 - DNA, 19 bp, Artificial Sequence
  Description of Artificial Sequence  Synthetic Primer
tgggatggagaagtgggat

SEQ ID NO: 11 - DNA, 24 bp, Artificial Sequence
  Description of Artificial Sequence  Synthetic Primer
tgggatggagaagtgggatccaaa

SEQ ID NO: 12 - DNA, 30 bp, Artificial Sequence
  Description of Artificial Sequence  Synthetic Primer
tgggatggagaagtgggaccaaagggagaa

SEQ ID NO: 13 - DNA, 20 bp, Artificial Sequence
  Description of Artificial Sequence  Synthetic Primer
agaatggaaaatcccagaaa

SEQ ID NO: 14 - DNA, 13 bp, Artificial Sequence
  Description of Artificial Sequence  Synthetic Primer
ttctcccgtttgg

SEQ ID NO: 15 - DNA, 7 bp, Artificial Sequence
  Description of Artificial Sequence  Synthetic Primer
ttctccc

SEQ ID NO: 16 - DNA, 20 bp, Artificial Sequence
  Description of Artificial Sequence  Synthetic Primer
ggatggagaaggtgggatcc

SEQ ID NO: 17 - DNA, 16 bp, Artificial Sequence
  Description of Artificial Sequence  Synthetic Primer
ccaaatgggagaattt

SEQ ID NO: 18 - DNA, 50 bp, Artificial Sequence
  Description of Artificial Sequence  Synthetic Primer
cacgaagaggccaagaaacagatcaacgattacgtggagaagggtactca

SEQ ID NO: 19 - DNA, 49 bp, Artificial Sequence
  Description of Artificial Sequence  Synthetic Primer
cacgaagaggccaagaaacagatcaacgattagtggagaagggtactca

SEQ ID NO: 20 - DNA, 60 bp, Artificial Sequence
  Description of Artificial Sequence  Synthetic Primer
ccctggcccacgaccactacaccatcgccgtccgcaacacccgccttgtgggcaaggagg

SEQ ID NO: 21 - DNA, 56 bp, Artificial Sequence
  Description of Artificial Sequence  Synthetic Primer
ccctggcccacgaccactacaccatcgccgtccactacaccgtcgccgtccgcaac

SEQ ID NO: 22 - DNA, 48 bp, Artificial Sequence
  Description of Artificial Sequence  Synthetic Primer
atctgtatgtttattgtaatgtcttcttcctcactcaccatagagtca

SEQ ID NO: 23 - DNA, 49 bp, Artificial Sequence
  Description of Artificial Sequence  Synthetic Primer
atctgtatgtttattgtaatgtcttcttcctcactcagagtcagatgta

SEQ ID NO: 24 - DNA, 47 bp, Artificial Sequence
  Description of Artificial Sequence  Synthetic Primer
cccacccttaggctgctggtggtctacccttggacccagaggttctt

SEQ ID NO: 25 - DNA, 45 bp, Artificial Sequence
  Description of Artificial Sequence  Synthetic Primer
cccacccttaggctgctggtggtctacccttggacagaggttctt